(19) United States
(12) Patent Application Publication
(10) Pub. No.: US 2007/0269818 A1
(43) Pub. Date: Nov. 22, 2007

(54) CARBOHYDRATE ARRAYS

(75) Inventor: Michael P. Savage, Sunnyvale, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/616,082

(22) Filed: Dec. 26, 2006

Related U.S. Application Data
(60) Provisional application No. 60/754,531, filed on Dec. 28, 2005.

(51) Int. Cl. C12Q 1/68

(52) U.S. Cl. 435/6

(57) ABSTRACT

Methods of detecting a carbohydrate binding compound in a sample by providing a high density oligonucleotide array including a plurality of probe sequences, hybridizing a plurality of carbohydrates that include an oligonucleotide that is complementary to a probe sequence to the high density oligonucleotide array, hybridizing a sample including a plurality of carbohydrate binding compounds to the high density oligonucleotide array, and detecting hybridization of at least one carbohydrate binding compound to at least one carbohydrate to determine the presence of the carbohydrate binding compound in a sample are provided.

### FIELD OF THE INVENTION

The present invention relates to carbohydrate arrays useful for detecting carbohydrate binding compounds.

### BACKGROUND OF THE INVENTION

Carbohydrates play important structural and functional roles in numerous physiological processes, including a variety of disease states such as cancer, bacterial infection, viral infection and inflammation (Koeller and Wong (2000) Glycobiology 10:1157). The study of carbohydrate binding compounds has been an area of keen interest in the fields of cell signaling and protein function for decades. Unfortunately, carbohydrate samples are expensive, often costing thousands of dollars per microgram. Currently, such expense has severely limited the widespread study of carbohydrate-carbohydrate binding protein interactions.

### SUMMARY OF THE INVENTION

Embodiments of the present invention are based in part on the discovery that a carbohydrate-nucleoside array can effectively be used to provide an economical and facile method to analyze carbohydrate binding to the carbohydrate binding compounds described herein. The arrays and methods described herein permit the analysis of as little as a picogram of carbohydrate while facilitating optimum usage of a carbohydrate sample.

The present invention provides methods of detecting a carbohydrate binding compound in a sample using a high density oligonucleotide array having a plurality of probes attached thereto to which a plurality of carbohydrate-oligonucleotides are hybridized. In certain embodiments, a method of the invention includes providing a high density oligonucleotide array comprising a plurality of probe sequences, hybridizing a plurality of carbohydrates that comprise an oligonucleotide that is complementary to a probe sequence to said high density oligonucleotide array, hybridizing a sample comprising a plurality of carbohydrate binding compounds to said high density oligonucleotide array, and detecting hybridization of at least one carbohydrate binding compound to at least one carbohydrate to determine the presence of the carbohydrate binding compound in a sample. In accordance with certain aspects, the plurality of carbohydrates is a plurality of substantially identical carbohydrates or a plurality of different carbohydrates. In yet another aspect, the sample is from a human, a cellular lysate or an in vitro translation reaction.

In accordance with certain aspects of the invention, the carbohydrate binding compound is a protein or polypeptide. In accordance with other aspects, the protein or polypeptide comprises a detectable label. In accordance with still other aspects, the protein or polypeptide is identified by antibody binding, mass spectroscopy or Edman degradation.

In accordance with certain aspects of the invention, the plurality of carbohydrates is released from the array. In accordance with other aspects, the releasing step is performed by contacting the array with an agent that increases hybridization stringency.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used herein, the singular forms “a,” “an,” and “the” include, but are not limited to, plural references unless the context clearly dictates otherwise. For example, the term “an agent” includes, but is not limited to, a plurality of agents, including mixtures thereof.

An individual is not limited to a human being, but may also be other organisms including, but not limited to, mammals, plants, bacteria, viruses and the like, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5 and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry and immunology, which are within the skill of the art. Such conventional techniques include polymer array synthesis, hybridization, ligation, and detection of hybridization using a label. Specific illustrations of suitable techniques can be had by reference to the description provided below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5th Ed., W.H. Freeman Pub., New York, N.Y., all of which are herein incorporated in their entirety by reference for all purposes.

The present invention can employ solid substrates, including arrays in certain embodiments. Methods and techniques applicable to polymer array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), each of which is incorporated herein by reference in its entirety for all purposes.

Patents that describe synthesis techniques in specific embodiments include U.S. Pat. Nos. 5,412,087, 6,147,205, 6,262,216, 6,310,189, 5,889,165 and 5,959,098, each of which is incorporated herein by reference in its entirety for all purposes. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GENECHIP®. Example arrays are shown on the website at affymetrix.com, incorporated herein by reference in its entirety for all purposes. Certain embodiments of the invention are directed to the use of high density oligonucleotide arrays. Thus, this invention provides for a method of simultaneously monitoring the expression (e.g. detecting and or quantifying the expression) of a multiplicity of carbohydrates. Preferably, at least about 1 carbohydrate, at least about 10 carbohydrates, more preferably at least about 100 carbohydrates, more preferably at least about 1000 carbohydrates, even more preferably at least about 10,000 carbohydrates are assayed at one time.

The following definitions are used, unless otherwise described.

The term “carbohydrate,” as used herein includes, but is not limited to, compounds that contain oxygen, hydrogen and carbon atoms, typically (C.H₂O)ₙ wherein n≧3. Carbohydrates include, but are not limited to, compounds such as monosaccharides, oligosaccharides, polysaccharides, glycoproteins, glycolipids and the like. Carbohydrates of the present invention include carbohydrate-nucleoside hybrid molecules, such as carbohydrate-oligonucleotide hybrid molecules.

As used herein, the term “monosaccharide” includes, but is not limited to, a compound that is the basic unit of a carbohydrate, consisting of a single sugar. Monosaccharides include, but are not limited to, glucose, glyceraldehydes, ribose, mannose, galactose and the like.

As used herein, the term “oligosaccharide” refers without limitation to several (e.g., two to ten) covalently linked monosaccharide units. Oligosaccharides include, but are not limited to, disaccharides (i.e., two monosaccharide units) such as sucrose, lactose, maltose, isomaltose, cellobiose and the like. Oligosaccharides are often associated with proteins (i.e., glycoproteins) and lipids (i.e., glycolipids). Oligosaccharides form two types of attachments to proteins: N-glycosidic, i.e., β linked to the amide nitrogen of an Asn in the sequence Asn-X-Ser or Asn-X-Thr, where X is typically any amino acid residue except Pro or Asp; and O-glycosidic, i.e., the most common attachment of which involves the disaccharide core β-galactosyl-(1→3)-α-N-acetylgalactosamine α linked to the OH group of either Ser or Thr. Less commonly, galactose, mannose or xylose can form α-O-glycosides with Ser or Thr. Galactose can also form O-glycosidic bonds to the hydroxylsyl residues of collagen.

As used herein, the term “polysaccharide” refers without limitation to many (e.g., eleven or more) covalently linked monosaccharide units. Polysaccharides can have molecular masses ranging well into millions of daltons. Polysaccharides include, but are not limited to, cellulose, chitin, starch, glycogen, glycosaminoglycans (e.g., hyaluronic acid, chondroitin-4-sulfate, chondroitin-6-sulfate, dermatan sulfate, keratin sulfate, heparin and the like) and the like.

As used herein, the term “carbohydrate binding compound” includes, but is not limited to, a compound that binds to a carbohydrate moiety such as a carbohydrate binding protein, e.g., a lectin or a galectin, a glycoprotein, a lipid, a nucleic acid (e.g., DNA or RNA), a small molecule or a portion thereof, such as, for example, a carbohydrate recognition domain.

As used herein, the term “lectin” includes, but is not limited to, any of a group of hemagglutinating proteins which bind specifically to the branching sugar molecules of glycoproteins and glycolipids on the surface of cells. Certain lectins selectively cause agglutination of erythrocytes of certain blood groups and of malignant cells but not their normal counterparts, while others stimulate the proliferation of lymphocytes.

As used herein, the term “galectin” refers to a general name proposed in 1994 for a family of animal lectins (Barondes et al. (1994) Cell 76:597). The term “galectin” refers without limitation to a lectin that has a galactose-binding ability as well as a galectin-specific amino acid sequence.

The term “nucleic acid,” as used herein includes, but is not limited to, a polymer comprising two or more nucleotides and includes single-, double- and triple stranded polymers. As used herein, the term “nucleotide” refers to both naturally occurring and non-naturally occurring compounds and comprises a heterocyclic base, a sugar, and a linking group, preferably a phosphate ester. As used herein, the term “nucleoside” refers to both naturally occurring and non-naturally occurring compounds and comprises a heterocyclic base and a sugar.

Structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

The term “oligonucleotide,” sometimes referred to as “polynucleotide,” includes, but is not limited to, a nucleic acid ranging from at least 5, 10, or 20 bases long and may be up to 20, 50, 100, 1,000, or 5,000 bases long and/or a compound that specifically hybridizes to a polynucleotide. A polymorphic site can occur within any position of the oligonucleotide. Oligonucleotides of the present invention include sequences of deoxyribonucleic acid (DNA) or ribonucleic acid (RNA) and mimetics thereof which may be isolated from natural sources, recombinantly produced or artificially synthesized. A further example of a polynucleotide of the present invention may be peptide nucleic acid (PNA). See U.S. Pat. No. 6,156,501, incorporated herein by reference in its entirety for all purposes. The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The phrase “coupled to a support” includes, but is not limited to, being bound directly or indirectly thereto including attachment by covalent binding, hydrogen bonding, ionic interaction, hydrophobic interaction, or otherwise.

The term “probe” includes, but is not limited to, a nucleic acid that can be used to detect, by hybridization, a target nucleic acid. Preferably, the probe is complementary to the target nucleic acid along the entire length of the probe, but hybridization can occur in the presence of one or more base mismatches between probe and target or in the presence of one or more universal base analogs. A probe includes a surface-immobilized molecule that can be recognized by a particular target. See U.S. Pat. No. 6,582,908, incorporated herein by reference in its entirety for all purposes, for an example of arrays having all possible combinations of probes with 10, 12 or more bases.

“Perfect match probe” includes, but is not limited to, a probe that has a sequence that is perfectly complementary to a particular target sequence. The test probe is typically perfectly complementary to a portion (subsequence) of the target sequence. The perfect match (PM) probe can be a “test probe,” a “normalization control” probe, an expression level control probe and the like. A perfect match control or perfect match probe is, however, distinguished from a “mismatch control” or “mismatch probe.” In the case of expression monitoring arrays, perfect match probes are typically preselected (designed) to be complementary to particular sequences or subsequences of target nucleic acids (e.g., particular genes). In contrast, in generic difference screening arrays, the particular target sequences are typically unknown. In the latter case, perfect match probes cannot be preselected. The term perfect match probe in this context is to distinguish that probe from a corresponding “mismatch control” that differs from the perfect match in one or more particular preselected nucleotides as described below.

“Mismatch control” or “mismatch probe,” in expression monitoring arrays, includes, but is not limited to, probes whose sequence is deliberately selected not to be perfectly complementary to a particular target sequence. For each mismatch (MM) control in a high-density array there preferably exists a corresponding perfect match (PM) probe that is perfectly complementary to the same particular target sequence. In “generic” (e.g., random, arbitrary, haphazard, etc.) arrays, since the target nucleic acid(s) are unknown, perfect match and mismatch probes cannot be a priori determined, designed, or selected. In this instance, the probes are preferably provided as pairs where each pair of probes differ in one or more preselected nucleotides. Thus, while it is not known a priori which of the probes in the pair is the perfect match, it is known that when one probe specifically hybridizes to a particular target sequence, the other probe of the pair will act as a mismatch control for that target sequence. It will be appreciated that the perfect match and mismatch probes need not be provided as pairs, but may be provided as larger collections (e.g., 3, 4, 5, or more) of probes that differ from each other in particular preselected nucleotides. While the mismatcn(s) may be located anywhere in the mismatch probe, terminal mismatches are less desirable as a terminal mismatch is less likely to prevent hybridization of the target sequence. In certain embodiments, the mismatch is located at or near the center of the probe such that the mismatch is most likely to destabilize the duplex with the target sequence under the test hybridization conditions. In other embodiments, perfect matches differ from mismatch controls in a single centrally-located nucleotide.

The terms “target nucleic acid” and “target oligonucleotide” refer in a non-limiting manner to a nucleic acid to which the probe is designed to specifically hybridize. In preferred aspects of the invention, the target nucleic acid or target oligonucleotide is associated with a carbohydrate. The target nucleic acid or target oligonucleotide has a sequence that is complementary to the nucleic acid sequence of the corresponding probe directed to the target. The terms target nucleic acid and target oligonucleotide may refer to the specific subsequence of a larger nucleic acid to which the probe is directed.

The terms “solid support,” “support,” and “substrate” as used herein are used interchangeably and include, but are not limited to, a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305, incorporated herein by reference in its entirety for all purposes, for exemplary substrates.

A preferred embodiment of the present invention provides a method for identifying a carbohydrate binding compound (e.g., a protein, peptide, lipid, peptidomimetic, small molecule, drug or the like) which binds to immobilized carbohydrates as described herein. In certain aspects, a carbohydrate binding compound is identified in a method wherein a sample is contacted to an array having carbohydrate-oligonucleotides attached thereto, and a carbohydrate binding compound present in the sample that binds to a carbohydrate attached to the array is detected.

One of skill in the art will appreciate that in order to analyze binding of a carbohydrate binding compound to a carbohydrate, it is desirable to provide a sample comprising one or more carbohydrate binding compounds, such as, for example, a biological sample, a sample from an in vitro translation reaction, a sample from a combinatorial library or the like.

The term “sample,” as used herein, includes a biological sample, which is intended to include, but is not limited to: tissues, cells and biological fluids isolated from a subject; tissues, cells and fluids present within a subject; as well as tissues, cells and biological fluids isolated from a subject and maintained in culture. Biological samples may be of any biological tissue or fluid or cells. Typical biological samples include, “clinical samples” which include, but are not limited to, sputum, lymph, blood, blood cells (e.g., white cells), fat cells, cervical cells, cheek cells, throat cells, mammary cells, muscle cells, skin cells, liver cells, spinal cells, bone marrow cells, tissue (e.g., muscle tissue, cervical tissue, skin tissue, spinal tissue, liver tissue and the like) fine needle biopsy samples, urine, cerebrospinal fluid, peritoneal fluid and pleural fluid, or cells therefrom. Biological samples may also include sections of tissues such as frozen sections taken for histological purposes or formalin-fixed, paraffin-embedded tissue. A biological sample may be obtained from a mammal, including, but not limited to horses, cows, sheep, pigs, goats, rabbits, guinea pigs, rats, mice, gerbils, non-human primates and humans. Biological samples may also include cells from microorganisms (e.g., bacterial cells, viral cells, yeast cells and the like) and portions thereof. As used herein, the term “biological fluid” is intended to include any fluid taken from a biological organism. Biological fluids include, but are not limited to, sputum, lymph, blood, urine, tears, breast milk, nipple aspirate fluid, seminal fluid, vaginal secretions, cerebrospinal fluid, peritoneal fluid, pleural fluid, pus, ascites and the like. Biological samples may also include sections of tissues such as frozen sections taken for histological purposes.

In one aspect, a biological sample is contacted to an array of the invention and the ability of a carbohydrate binding compound to bind to the array, i.e., to an immobilized carbohydrate, is determined. Methods for detecting such complexes include immunodetection of complexes using antibodies reactive with carbohydrate binding compound, as well as enzyme-linked assays which rely on detecting an enzymatic activity associated with the carbohydrate binding compound, or identifying the carbohydrate binding compound using a variety of analytical methods such as mass spectroscopy, Edman degradation, nuclear magnetic resonance, liquid chromatography/mass spectroscopy and the like (See Steen and Mann (2004) Nature Reviews Molecular Cell Biology 5:699; Alberts et al. (1998) Essential Cell Biology. An Introduction to the Molecular Biology of the Cell. Garland Publishing, New York; each of which is incorporated herein by reference in its entirety for all purposes).

Detectable labels suitable for use in certain aspects of the present invention include any composition detectable by spectroscopic, photochemical, biochemical, immunochemical, electrical, optical or chemical means. Useful labels in accordance with an aspect of the present invention include biotin for staining with labeled streptavidin conjugate, magnetic beads (e.g., DYNABEADS™), fluorescent dycs (e.g., yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like), luminescent and bioluminescent markers (e.g., biotin, luciferase (e.g., bacterial, firefly, click beetle and the like), luciferin, aequorin and the like), radiolabels (e.g., ³H, ¹²⁵I, ³⁵S, ¹⁴C, or ³²P), enzymes (e.g., galactosidases, glucorinidases, phosphatases (e.g., alkaline phosphatase), peroxidases (e.g., horseradish peroxidase), cholinesterases and the like), and calorimetric labels such as colloidal gold or colored glass or plastic (e.g., polystyrene, polypropylene, latex, and the like) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference in its entirety for all purposes.

Means of detecting such labels are well known to those of skill in the art. Thus, for example, radiolabels may be detected using photographic film or scintillation counters, fluorescent markers may be detected using a photo detector to detect emitted light. Enzymatic labels are typically detected by providing the enzyme with a substrate and detecting the reaction product produced by the action of the enzyme on the substrate, and calorimetric labels are detected by simply visualizing the colored label.

In accordance with an aspect of the present invention, the label may be added to the carbohydrate binding compound prior to, or after the hybridization. “Direct labels,” as used herein, are detectable labels that are directly attached to or incorporated into the carbohydrate binding compound nucleic acid prior to hybridization. In contrast, “indirect labels,” as used herein, are joined to the hybridized carbohydrate binding compound after hybridization. Often, the indirect label is attached to a binding moiety that has been attached to the carbohydrate binding compound prior to the hybridization. Thus, for example, the target carbohydrate binding compound may be biotinylated before the hybridization. After hybridization, an avidin-conjugated fluorophore will bind the biotin bearing hybrid duplexes providing a label that is easily detected.

In a preferred embodiment, the present invention provides an array of oligonucleotide probes. In certain aspects, an array of oligonucleotide probes is a high density array comprising greater than about 100, greater than about 1,000, greater than about 16,000, or greater than about 65,000 or 250,000 or even 1,000,000 different oligonucleotide probes. Such high density arrays comprise a probe density of generally greater than about 60, more generally greater than about 100, most generally greater than about 600, often greater than about 1000, more often greater than about 5,000, most often greater than about 10,000, greater than about 40,000, greater than about 100,000, or greater than about 400,000 different oligonucleotide probes per cm². The oligonucleotide probes range from about 5 to about 50 nucleotides, from about 10 to about 40 nucleotides, or from about 15 to about 40 nucleotides in length. The array may comprise more than 10, more than 50, more than 100, or more than 1000 oligonucleotide probes specific for each target nucleic acid-carbohydrate complex. Although a planar array surface typically used, the array may be fabricated on a surface of virtually any shape or even a multiplicity of surfaces. High density arrays of the invention are further described in U.S. Pat. No. 6,040,138, incorporated herein by reference in its entirety for all purposes.

One of skill in the art will appreciate that an enormous number of array designs are suitable for the practice of this invention. The high density array will typically include a number of probes that specifically hybridize to the nucleic acid(s) associated with carbohydrate(s). In addition, in a preferred embodiment, the array will include one or more control probes.

In its simplest embodiment, the high density array includes “test probes.” These are oligonucleotides that range from about 5 to about 45 or 5 to about 50 nucleotides, more preferably from about 10 to about 40 nucleotides and most preferably from about 15 to about 40 nucleotides in length. In other particularly preferred embodiments the probes are 20 or 25 nucleotides in length. These oligonucleotide probes have sequences complementary to particular oligonucleotides, such as those associated with a carbohydrate. Thus, the test probes are capable of specifically hybridizing to the target oligonucleotide.

In addition to test probes that bind the target oligonucleotide, the high density array can contain a number of control probes. The control probes fall into three categories referred to herein as 1) normalization controls; 2) expression level controls; and 3) mismatch controls.

Normalization controls are oligonucleotide probes that are perfectly complementary to labeled reference oligonucleotides that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g., fluorescence intensity) read from all other probes in the array are divided by the signal (e.g., fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few normalization probes are used and they are selected such that they hybridize well (i.e. no secondary structure) and do not match any target-specific probes.

Normalization probes can be localized at any position in the array or at multiple positions throughout the array to control for spatial variation in hybridization efficiently. In a preferred embodiment, the normalization controls are located at the corners or edges of the array as well as in the middle.

Expression level controls are probes that hybridize specifically with constitutively expressed genes in the biological sample. Expression level controls are designed to control for the overall health and metabolic activity of a cell. Examination of the co-variance of an expression level control with the expression level of a target carbohydrate binding compound indicates whether measured changes or variations in expression level of a gene is due to changes in transcription rate of that gene or to general variations in health of the cell. Thus, for example, when a cell is in poor health or lacking a critical metabolite the expression levels of both an active target gene and a constitutively expressed gene are expected to decrease. The converse is also true. Thus, where the expression levels of both an expression level control and the target carbohydrate binding compound appear to both decrease or to both increase, the change may be attributed to changes in the metabolic activity of the cell as a whole, not to differential expression of the target carbohydrate binding compound in question. Conversely, where the expression levels of the target carbohydrate binding compound and the expression level control do not co-vary, the variation in the expression level of the target carbohydrate binding compound is attributed to differences in regulation of that compound and not to overall variations in the metabolic activity of the cell.

Virtually any constitutively expressed gene provides a suitable target for expression level controls. Typically expression level control probes have sequences complementary to subsequences of constitutively expressed “housekeeping genes” including, but not limited to the β-actin gene, the transferrin receptor gene, the GAPDH gene, and the like.

Mismatch controls may also be provided for the probes to the target genes, for expression level controls or for normalization controls. Mismatch controls are oligonucleotide probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a 20-mer, a corresponding mismatch probe will have the identical sequence except for a single base mismatch (e.g., substituting a G, a C or a T for an A) at any of positions 6 though 14 (the central mismatch). Mismatch probes thus provide a control for non-specific binding or cross-hybridization to a nucleic acid in the sample other than the target to which the probe is directed. Mismatch probes thus indicate whether a hybridization is specific or not.

In a preferred embodiment, oligonucleotide probes in the high density array are selected to bind specifically to the oligonucleotide target to which they are directed with minimal non-specific binding or cross-hybridization under the particular hybridization conditions utilized. Because the high density arrays of this invention can contain in excess of 1,000,000 different probes, it is possible to provide every probe of a characteristic length that binds to a particular nucleic acid sequence.

However, certain probes simply may not hybridize effectively under the hybridization conditions (e.g., due to secondary structure, or interactions with the substrate or other probes). Thus, in a preferred embodiment, the probes that show such poor specificity or hybridization efficiency are identified and may not be included either in the high density array itself (e.g., during fabrication of the array) or in the post-hybridization data analysis.

Methods of forming high density arrays of oligonucleotides, peptides and other polymer sequences with a minimal number of synthetic steps are known. An oligonucleotide analogue array of the invention can be synthesized on a solid substrate by a variety of methods, including, but not limited to, light-directed chemical coupling, and mechanically directed coupling. See U.S. Pat. No. 5,143,854, PCT Publication Nos. WO 90/15070, WO 92/10092 and WO 93/09668, U.S. patent application Ser. Nos. 07/980,523 and 08/082,937, and Fodor et al. (1991) Science 251:767, which disclose methods of forming vast arrays of peptides, oligonucleotides and other molecules using, for example, light-directed synthesis techniques, each of which is incorporated herein by reference in its entirety for all purposes. These procedures for synthesis of polymer arrays are referred to as VLSIPS™ procedures. Using the VLSIPS™ approach, one heterogeneous array of polymers is converted, through simultaneous coupling at a number of reaction sites, into a different heterogeneous array. See, U.S. application Ser. Nos. 07/796,243 and 07/980,523, incorporated herein by reference in their entirety for all purposes.

The development of VLSIPS™ technology as described in the above-noted U.S. Pat. No. 5,143,854 and PCT Patent Publication Nos. WO 90/15070 and 92/10092, is considered pioneering technology in the fields of combinatorial synthesis and screening of combinatorial libraries. In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In the event that an oligonucleotide analogue with a polyamide backbone is used in the VLSIPS™ procedure, it is generally inappropriate to use phosphoramidite chemistry to perform the synthetic steps, since the monomers do not attach to one another via a phosphate linkage. Instead, peptide synthetic methods are substituted. See, e.g., U.S. Pat. No. 5,143,854, incorporated herein by reference in its entirety for all purposes.

Peptide nucleic acids comprise a polyamide backbone and the bases found in naturally occurring nucleoside. Such nucleic acid are commercially available from, e.g., Biosearch, Inc. (Bedford, Mass.). Peptide nucleic acids are capable of binding to nucleic acids with high specificity, and are considered “oligonucleotide analogues” for purposes of this disclosure.

In addition to the foregoing, aspects of the invention include additional methods which can be used to generate an array of oligonucleotides on a single substrate are described in U.S. Pat. Nos. 5,677,195 and 5,384,261, and in PCT Publication No. WO 93/09668, each of which is incorporated herein by reference in its entirety for all purposes. In the methods disclosed in these applications, reagents are delivered to the substrate by either (1) flowing within a channel defined on predefined regions or (2) “spotting” on predefined regions or (3) through the use of photoresist. However, other approaches, as well as combinations of spotting and flowing, may be employed. In each instance, certain activated regions of the substrate are mechanically separated from other regions when the monomer solutions are delivered to the various reaction sites.

In one aspect, a typical “flow channel” method is applied to the compounds and libraries of the present invention, and can generally be described as follows. Diverse polymer sequences are synthesized at selected regions of a substrate or solid support by forming flow channels on a surface of the substrate through which appropriate reagents flow or in which appropriate reagents are placed. For example, assume a monomer “A” is to be bound to the substrate in a first group of selected regions. If necessary, all or part of the surface of the substrate in all or a part of the selected regions is activated for binding by, for example, flowing appropriate reagents through all or some of the channels, or by washing the entire substrate with appropriate reagents. After placement of a channel block on the surface of the substrate, a reagent having the monomer A flows through or is placed in all or some of the channel(s). The channels provide fluid contact to the first selected regions, thereby binding the monomer A on the substrate directly or indirectly (via a spacer) in the first selected regions.

Thereafter, a monomer B is coupled to second selected regions, some of which may be included among the first selected regions. The second selected regions will be in fluid contact with a second flow channel(s) through translation, rotation, or replacement of the channel block on the surface of the substrate; through opening or closing a selected valve; or through deposition of a layer of chemical or photoresist. If necessary, a step is performed for activating at least the second regions. Thereafter, the monomer B is flowed through or placed in the second flow channel(s), binding monomer B at the second selected locations. In this particular example, the resulting sequences bound to the substrate at this stage of processing will be, for example, A, B, and AB. The process is repeated to form a vast array of sequences of desired length at known locations on the substrate.

After the substrate is activated, monomer A can be flowed through some of the channels, monomer B can be flowed through other channels, a monomer C can be flowed through still other channels, etc. In this manner, many or all of the reaction regions are reacted with a monomer before the channel block must be moved or the substrate must be washed and/or reactivated. By making use of many or all of the available reaction regions simultaneously, the number of washing and activation steps can be minimized.

One of skill in the art will recognize that there are alternative methods of forming channels or otherwise protecting a portion of the surface of the substrate. For example, according to some embodiments, a protective coating such as a hydrophilic or hydrophobic coating (depending upon the nature of the solvent) is utilized over portions of the substrate to be protected, sometimes in combination with materials that facilitate wetting by the reactant solution in other regions. In this manner, the flowing solutions are further prevented from passing outside of their designated flow paths.

In another aspect, the “spotting” methods of preparing compounds and libraries of the present invention can be implemented in much the same manner as the flow channel methods. For example, a monomer A can be delivered to and coupled with a first group of reaction regions which have been appropriately activated. Thereafter, a monomer B can be delivered to and reacted with a second group of activated reaction regions. Unlike the flow channel embodiments described above, reactants are delivered by directly depositing (rather than flowing) relatively small quantities of them in selected regions. In some steps, of course, the entire substrate surface can be sprayed or otherwise coated with a solution. In preferred embodiments, a dispenser moves from region to region, depositing only as much monomer as necessary at each stop. Typical dispensers include a micropipette to deliver the monomer solution to the substrate and a robotic system to control the position of the micropipette with respect to the substrate. In other embodiments, the dispenser includes a series of tubes, a manifold, an array of pipettes, or the like so that various reagents can be delivered to the reaction regions simultaneously.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency in this case in 6×SSPE-T at 37° C. (0.005% Triton X-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g., down to as low as 0.25×SSPE-T at 37° C. to 50° C.) until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

In a preferred embodiment, background signal is reduced by the use of a detergent (e.g., C-TAB) or a blocking reagent (e.g., sperm DNA, cot-1 DNA, etc.) during the hybridization to reduce non-specific binding. In a particularly preferred embodiment, the hybridization is performed in the presence of about 0.5 mg/ml DNA (e.g., herring sperm DNA). The use of blocking agents in hybridization is well known to those of skill in the art (see, e.g., Chapter 8 in P. Tijssen, supra)

The stability of duplexes formed between RNAs or DNAs are generally in the order of RNA:RNA>RNA:DNA>DNA:DNA in solution. Long probes have better duplex stability with a target, but poorer mismatch discrimination than shorter probes (mismatch discrimination refers to the measured hybridization signal ratio between a perfect match probe and a single base mismatch probe). Shorter probes (e.g., 8-mers) discriminate mismatches very well, but the overall duplex stability is low.

Altering the thermal stability (Tₘ) of the duplex formed between the target and the probe using, e.g., known oligonucleotide analogues allows for optimization of duplex stability and mismatch discrimination. One useful aspect of altering the Tₘ arises from the fact that adenine-thymine (A-T) duplexes have a lower Tₘ than guanine-cytosine (G-C) duplexes, due in part to the fact that the A-T duplexes have 2 hydrogen bonds per base-pair, while the G-C duplexes have 3 hydrogen bonds per base pair. In heterogeneous oligonucleotide arrays in which there is a non-uniform distribution of bases, it is not generally possible to optimize hybridization for each oligonucleotide probe simultaneously. Thus, in some embodiments, it is desirable to selectively destabilize G-C duplexes and/or to increase the stability of A-T duplexes. This can be accomplished, e.g., by substituting guanine residues in the probes of an array which form G-C duplexes with hypoxanthine, or by substituting adenine residues in probes which form A-T duplexes with 2,6 diaminopurine or by using the salt tetramethyl ammonium chloride (TMAC1) in place of NaCl.

Altered duplex stability conferred by using oligonucleotide analogue probes can be ascertained by following, e.g., fluorescence signal intensity of oligonucleotide analogue arrays hybridized with a target oligonucleotide over time. The data allow optimization of specific hybridization conditions at, e.g., room temperature (for simplified diagnostic applications in the future).

Another way of verifying altered duplex stability is by following the signal intensity generated upon hybridization with time. Previous experiments using DNA targets and DNA chips have shown that signal intensity increases with time, and that the more stable duplexes generate higher signal intensities faster than less stable duplexes. The signals reach a plateau or “saturate” after a certain amount of time due to all of the binding sites becoming occupied. These data allow for optimization of hybridization, and determination of the best conditions at a specified temperature.

Methods of optimizing hybridization conditions are well known to those of skill in the art (see, e.g., Laboratory Techniques in Biochemistry and Molecular Biology, Vol. 24: Hybridization With Nucleic Acid Probes, P. Tijssen, ed. Elsevier, N.Y., (1993), incorporated herein by reference in its entirety for all purposes).

Means of detecting labeled target nucleic acids hybridized to the probes of the high density array are known to those of skill in the art. Thus, for example, where a calorimetric label is used, simple visualization of the label is sufficient. Where a radioactive labeled target nucleic acid is used, detection of the radiation (e.g., with photographic film or a solid state detector) is sufficient.

In a preferred embodiment, the target nucleic acids are labeled with a fluorescent label and the localization of the label on the probe array is accomplished with fluorescent microscopy. The hybridized array is excited with a light source at the excitation wavelength of the particular fluorescent label and the resulting fluorescence at the emission wavelength is detected. In a particularly preferred embodiment, the excitation light source is a laser appropriate for the excitation of the fluorescent label.

The confocal microscope may be automated with a computer-controlled stage to automatically scan the entire high density array. Similarly, the microscope may be equipped with a phototransducer (e.g., a photomultiplier, a solid state array, a ccd camera, etc.) attached to an automated data acquisition system to automatically record the fluorescence signal produced by hybridization to each oligonucleotide probe on the array. Such automated systems are described at length in U.S. Pat. Nos. 5,143,854 and 5,631,734, and PCT Application 92/10092, each of which is incorporated herein by reference in its entirety for all purposes. Use of laser illumination in conjunction with automated confocal microscopy for signal detection permits detection at a resolution of better than about 100 μm, more preferably better than about 50 μm, and most preferably better than about 25 μm.

One of skill in the art will appreciate that methods for evaluating the hybridization results vary with the nature of the specific probe nucleic acids used as well as the controls provided. In the simplest embodiment, simple quantification of the fluorescence intensity for each target nucleic acid is determined. This is accomplished simply by measuring target nucleic acid signal strength at each location (representing a different carbohydrate) on the high density array (e.g., where the label is a fluorescent label, detection of the amount of florescence (intensity) produced by a fixed excitation illumination at each location on the array). Comparison of the absolute intensities of an array hybridized to target nucleic acids from a “test” sample with intensities produced by a “control” sample provides a measure of the relative expression of the nucleic acids that hybridize to each of the probes.

One of skill in the art, however, will appreciate that hybridization signals will vary in strength with efficiency of hybridization, the amount of label on the target nucleic acid and the amount of the particular target nucleic acid in the sample. Typically, target nucleic acids present at very low levels will show a very weak signal. At some low level of concentration, the signal becomes virtually indistinguishable from background. In evaluating the hybridization data, a threshold intensity value may be selected below which a signal is not counted as being essentially indistinguishable from background.

Where it is desirable to detect target nucleic acids expressed at lower levels, a lower threshold is chosen. Conversely, where only high expression levels are to be evaluated a higher threshold level is selected. In a preferred embodiment, a suitable threshold is about 10% above that of the average background signal.

In addition, the provision of appropriate controls permits a more detailed analysis that controls for variations in hybridization conditions, cell health, non-specific binding and the like. Thus, for example, in a preferred embodiment, the hybridization array is provided with normalization controls as described above. These normalization controls are probes complementary to control sequences added in a known concentration to the sample. Where the overall hybridization conditions are poor, the normalization controls will show a smaller signal reflecting reduced hybridization. Conversely, where hybridization conditions are good, the normalization controls will provide a higher signal reflecting the improved hybridization. Normalization of the signal derived from other probes in the array to the normalization controls thus provides a control for variations in hybridization conditions. Typically, normalization is accomplished by dividing the measured signal from the other probes in the array by the average signal produced by the normalization controls. Normalization may also include correction for variations due to sample preparation and amplification. Such normalization may be accomplished by dividing the measured signal by the average signal from the sample constant value to scale the results.

As indicated above, the high density array can include mismatch controls. In a preferred embodiment, there is a mismatch control having a central mismatch for every probe (except the normalization controls) in the array. It is expected that after washing in stringent conditions, where a perfect match would be expected to hybridize to the probe, but not to the mismatch, the signal from the mismatch controls should only reflect non-specific binding or the presence in the sample of a nucleic acid that hybridizes with the mismatch. Where both the probe in question and its corresponding mismatch control both show high signals, or the mismatch shows a higher signal than its corresponding test probe, there is a problem with the hybridization and the signal from those probes is ignored. The difference in hybridization signal intensity between the target specific probe and its corresponding mismatch control is a measure of the discrimination of the target-specific probe. Thus, in a preferred embodiment, the signal of the mismatch probe is subtracted from the signal from its corresponding test probe to provide a measure of the signal due to specific binding of the test probe.

The concentration of a particular sequence can then be determined by measuring the signal intensity of each of the probes that bind specifically to the target oligonucleotide and normalizing to the normalization controls. Where the signal from the probes is greater than a target oligonucleotide bound to a mismatch, the mismatch is subtracted. Where the intensity of the target oligonucleotide bound to the mismatch is equal to or greater than its corresponding test probe, the signal is ignored. The hybridization level of a particular target oligonucleotide can then be scored by the number of positive signals (either absolute or above a threshold value), the intensity of the positive signals (either absolute or above a selected threshold value), or a combination of both metrics (e.g., a weighted average).

Normalization controls are often unnecessary for useful quantification of a hybridization signal. Thus, where optimal probes have been identified in the two step selection process as described above, the average hybridization signal produced by the selected optimal probes provides a good quantified measure of the concentration of hybridized nucleic acid.

The methods of monitoring carbohydrate compound binding to the arrays of the invention may be performed utilizing a computer. The computer typically runs a software program that includes computer code incorporating the invention for analyzing hybridization intensities measured from a substrate or chip. Methods of this invention that may be performed utilizing a computer are described in U.S. Patent Application No. 20030186296, incorporated herein by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, etc. The computer-executable instructions may be written in a suitable computer language or combination of several languages. Basic computational biology methods are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics. Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108, each of which is incorporated herein by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170, each of which is incorporated herein by reference in its entirety for all purposes.

Additionally, the present invention may have preferred embodiments that include methods for providing biological information over networks such as the internet as shown in U.S. Ser. Nos. 10/197,621, 10/063,559 (United States Publication No. 20020183936), 10/065,856, 10/065,868, 10/328,818, 10/328,872, 10/423,403, and 60/482,389, each of which is incorporated herein by reference in its entirety for all purposes.

This invention is further illustrated by the following examples, which should not be construed as limiting. The contents of all references, patents and published patent applications cited throughout this application are hereby incorporated by reference in their entirety for all purposes.

### EXAMPLE I

### Carbohydrate-Carbohydrate Binding Compound Query

A carbohydrate having a specific activatable site can be provided. The carbohydrate can then be attached to a substrate (e.g., glass) via a linker molecule. In a certain aspect of the invention, a library of carbohydrate-oligonucleotide hybrids can be generated using, for example, a GENFLEX™ Tag Array (See Affymetrix.com) to generate a spatially addressable, custom array. For example, Tag A, Tag B and Tag C oligonucleotide sequences can be linked to a glass substrate. A carbohydrate associated linker comprising a complementary Tag′ oligonucleotide sequence that specifically hybridizes to a glass-linked Tag can then be hybridized. For example, a carbohydrate A-Tag A′ hybrid would hybridize to Tag A, a carbohydrate B-Tag B′ hybrid would hybridize to Tag B, a carbohydrate C-Tag C′ hybrid would hybridize to Tag C, and so forth. The array could then be contacted with one or more carbohydrate binding compounds and carbohydrate-carbohydrate binding compound interactions could be determined.

What is claimed is:

1. A method of detecting a carbohydrate binding compound in a sample comprising:
providing a high density oligonucleotide array comprising a plurality of probe sequences;
hybridizing a plurality of carbohydrates, each of which comprises an oligonucleotide that is complementary to a probe sequence, to said high density oligonucleotide array;
hybridizing a sample comprising a plurality of carbohydrate binding compounds to said high density oligonucleotide array; and
detecting hybridization of at least one carbohydrate binding compound to at least one carbohydrate to determine the presence of the carbohydrate binding compound in a sample.

2. The method of claim 1, wherein said carbohydrate binding compound is a protein or polypeptide.

3. The method of claim 2, wherein said protein or polypeptide comprises a detectable label.

4. The method of claim 2, wherein said protein or polypeptide is identified by antibody binding.

5. The method of claim 2, wherein said protein or polypeptide is identified by mass spectroscopy or Edman degradation.

6. The method of claim 1, further comprising releasing said plurality of carbohydrates from said array.

7. The method of claim 6, wherein said releasing is performed by contacting said high density oligonucleotide array with an agent that increases hybridization stringency.

8. The method of claim 1, wherein said plurality of carbohydrates is a plurality of substantially identical carbohydrates.

9. The method of claim 1, wherein said plurality of carbohydrates is a plurality of different carbohydrates.

10. The method of claim 1, wherein said sample is from a human.

11. The method of claim 1, wherein said sample is a cellular lysate.

12. The method of claim 1, wherein said sample is from an in vitro translation reaction.
